(19) United States
(12) Patent Application Publication
(10) Pub. No.: US 2006/0148804 A1
(43) Pub. Date: Jul. 6, 2006

(54) Pde4 inhibitors for the treatment of neoplasms of lymphoid cells

(76) Inventors: Jurgen Braunger, Konstanz (DE); Christian Schudt, Konstanz (DE)

Correspondence Address: NATH & ASSOCIATES PLLC, 112 South West Street, Alexandria, VA, 22314, US

(21) Appl. No.: 10/542,088

(22) PCT Filed: Jan. 14, 2004
(86) PCT No.: PCT/EP04/00196

§ 371 Date: Jul. 13, 2005

(30) Foreign Application Priority Data
Jan. 14, 2003 (EP) 030007876

(51) Int. Cl. A61K 31/502

(52) U.S. Cl. 514/248

(57) ABSTRACT

The invention relates to the use of certain PDE4 inhibitors alone or in combination with one or more differentiation inducing agents and/or an agent effective in raising intracellular concentrations of cAMP or a stable analogue of cAMP in the preparation of pharmaceutical compositions for the treatment of neoplasms of lymphoid cells.

### FIELD OF APPLICATION OF THE INVENTION

The present invention relates to the use of certain PDE4 inhibitors in the treatment of neoplasms of lymphoid cells.

### KNOWN TECHNICAL BACKGROUND

Neoplasms of lymphoid cells can present clinically as leukemia, lymphoma and myeloma.

Leukemias are classified as either lymphocytic or myeloid, depending on the type of leukocyte affected. In addition, leukemias are classified as either acute, referring to a rapidly progressing disease that involves immature leukocytes, or chronic, referring to a slower proliferation involving mature white cells. In acute leukemias, immature nonfunctioning leukocytes called blast cells proliferate.

The myeloid leukemias affect white blood cells (myelocytes) that give rise to granulocytes (phagocytic white blood cells that mount an inflammatory immune response). They include chronic myeloid leukemia (CML) and acute myeloid leukemia (AML), also called acute nonlymphocytic leukemia (ANLL).

The lymphocytic leukemias affect the white blood cells that give rise to various types of lymphocytes. They include acute lymphocytic leukemia (ALL); chronic lymphocytic leukemia (CLL), also called chronic granulocytic leukemia; and hairy cell leukemia (HCL), a chronic leukemia named for the cells' tiny hairlike projections. The lymphocytic leukemias are sometimes referred to as B cell leukemias or T cell leukemias depending upon whether they arise in antibody-producing B cells (HCL, CLL, and some cases of ALL) or in the T cell lymphocytes involved in cell-mediated immunity (some cases of ALL). Each of these types may be further classified into subtypes. Most childhood leukemias are of the acute lymphocytic type, acute myeloid leukemia is the most common type of adult leukemia.

The diagnosis of leukemia is confirmed by finding a disproportionate number of leukocytes in tissue obtained from a bone marrow biopsy. The course of treatment is based upon the type of cell affected, the progression of the disease, and the age of the patient.

Treatment may include chemotherapy with anticancer drugs, radiation therapy, blood and plasma transfusions, and bone marrow transplantation. In bone marrow transplantation, healthy bone marrow (either donated by a closely matched donor or treated marrow from the patent) is infused into the patient after the patient has undergone a course of marrow-destroying very high dose chemotherapy.

Recent studies have indicated that blood from a newborn infant's umbilical cord and placenta (called cord blood) can be used effectively instead of marrow transplants in some leukemias. Biological therapy (sometimes called immunotherapy) is also being introduced. Biological therapies include monoclonal antibodies, interferons, and maturation drugs, such as all-trans retinoic acid. These therapies may enhance the body's natural reaction to leukemia by bolstering the immune response or may encourage maturation of immature leukemic cells or reproduction of needed healthy blood elements.

Another more experimental approach suggests that agents capable of modulating 3′,5′-cyclic adenosine monophosphate (cAMP) levels might be useful for the treatment of lymphoid malignancies (Lerner A, Kim B, Lee R. Leuk Lymphoma 2000; 37:39-51). It has been published that elevated intracellular levels of cAMP can induce apoptosis in susceptible subpopulations of both B- and T-lineage lymphocytes. One means of augmenting cAMP signaling has been through the use of cAMP phosphodiesterase (PDE) inhibitors, as inhibition of cAMP catabolism results in elevation of intracellular lymphoid cAMP levels in vivo (Tohda Y, Nakahara H, Kubo H, Ohkawa K, Fukuoka M, Nakajima S. Gen. Pharmacol. 1998; 31: 409-13). Theophylline, a nonspecific methylxanthine PDE inhibitor, has been shown to induce apoptosis in chronic lymphocytic leukemia (CLL) B-lymphocytes in vitro (Mentz F, Merle-Beral H, Ouaaz F, Binet J-L. Br. J. Hematol. 1995; 90: 957-9; Mentz F, Mossalayi M D, Ouaaz F, Baudet S, Issaly F, Ktorza S, Semichon M, Binet J-L, Merle-Beral H. Blood 1996; 88: 2172-82). A subsequent Phase 2 clinical trial demonstrated that combined treatment with theophylline and chlorambucil induced positive responses in CLL patents who failed treatment with chlorambucil alone (Binet J-L, Mentz F, Leblond V, Merle-Beral H. Leukemia 1995; 9: 2159). Since theophylline is a nonselective PDE inhibitor as well as an adenosine receptor antagonist, this reagent complicates both the clinical and research applications. A more selective PDE inhibitor might also induce apoptosis in lymphoid cells and have therapeutic value in the treatment of lymphoid malignancies. Lymphoid cells contain several classes of cyclic nucleotide PDEs, including cGMP-inhibited PDE3 (Ekholm D, Hemmer B, Gao G, Vergelli M, Martin R, Manganiello V. J. Immunol. 1997; 159:1520-9) and cAMP-specific PDE4 (Erdogan S, Houslay M D. Biochem. J. 1997; 321:165-75).

Certain recently published scientific papers mention the potential use of PDE4 inhibitors in the induction of apoptosis in CLL cells (see for example: Kim, D. H. and Lerner A: “Type 4 cyclic adenosine monophosphate phosphodiesterase as a therapeutic target in chronic lymphocytic leukemia”, Blood, 92: 2484-2494, 1998; Lerner A., Kim B. and Lee R.: “The cAMP signalling pathway as a therapeutic target in lymphoid malignancies”. Leuk Lymphoma, 37: 39-51, 2000). In other publications it is de scribed that PDE4 inhibitors may also have therapeutic potential in human acute lymphoblastic leukemia (see for example: R. Ogawa, M. B. Streiff, A. Bugayenko and G. J. Kato: “Inhibition of PDE4 phosphodiesterase activity induces growth suppression, apoptosis, glucocorticoid sensitivity, p53 and p21^{WAF1/CIP1} proteins in human acute lymphoblastic leukemia cells”).

### DESCRIPTION OF THE INVENTION

It has been found that certain PDE4 inhibitors alone or in combination with differentiation inducing agents and/or cAMP agonists or stable analogs of cAMP are particularly useful in the treatment of neoplasms of lymphoid cells.

One class of PDE4 inhibitor compounds that may be usefully employed in the present invention includes compounds of formula 1 (embodiment A): 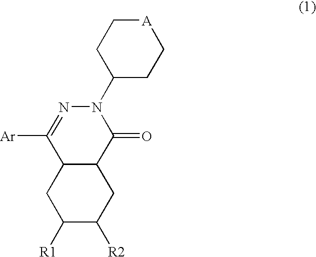
in which
R1 and R2 are both hydrogen or together form an additional bond,
A represents S (sulfur), S(O) (sulfoxide) or S(O)₂ (sulfone),
Ar represents a benzene derivative of formula (a) or (b) 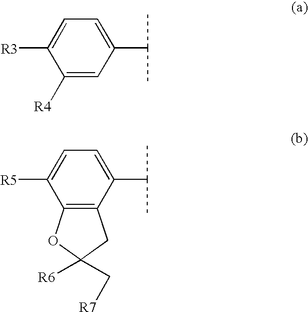
   wherein
R3 is halogen, 1-4C-alkoxy, or 1-4C-alkoxy which is completely or predominantly substituted by fluorine,
R4 is halogen, 1-8C-alkoxy, 3-7C-cycloalkoxy, 3-7C-cycloalkylmethoxy, or 1-4C-alkoxy which is completely or predominantly substituted by fluorine,
R5 is halogen, 1-4C-alkoxy, 3-5C-cycloalkoxy, 3-5C-cycloalkylmethoxy, or 1-4C-alkoxy which is completely or predominantly substituted by fluorine,
R6 is 1-4C-alkyl and
R7 is hydrogen or 14C-alkyl,
   or wherein
R6 and R7 together and with inclusion of the two carbon atoms, to which they are bonded, form a spiro-linked 5-, 6- or 7-membered hydrocarbon ring, optionally interrupted by an oxygen or sulphur atom,
   or the pharmaceutically acceptable salts thereof.

Compounds of embodiment A which are to be emphasized in this connection are those compounds of formula 1 in which
R1 and R2 together form an additional bond,
A represents S(O) (sulfoxide) or S(O)₂ (sulfone),
Ar represents a benzene derivative of formula (a) or (b) 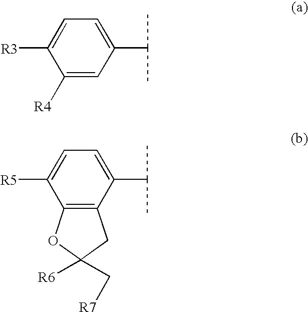
   wherein
R3 is 1-2C-alkoxy, or 1-2C-alkoxy which is completely or predominantly substituted by fluorine,
R4 is halogen, 1-4C-alkoxy or 3-5C-cycloalkoxy,
R5 is 1-2C-alkoxy, or 1-2C-alkoxy which is completely or predominantly substituted by fluorine,
R6 is methyl,
R7 is hydrogen,
   or wherein
R6 and R7 together and with inclusion of the two carbon atoms, to which they are bonded, form a spiro-linked cyclopentane or cyclohexane ring,
   or the pharmaceutically acceptable salts thereof.

Preferred compounds of embodiment A are in this connection compounds of formula 1 selected from
(cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one,
(cis)-4-(3,4-Dimethoxyphenyl)-2-(1,1-dioxohexahydro-1l⁶-thiopyranyl)-4a,5,8,8a-tetrahydro-2H-phthalazin-1-one,
(cis)-4-(3,4-Dimethoxyphenyl)-2-(1-oxo-hexahydro-1l⁴-thiopyran-4-yl)-4a,5,8,8a-tetrahydro-2H-phthalazin-1-one,
(cis)-4-(3-Chloro-4-methoxyphenyl)-2-(tetrahydrothiopyran-4-yl)-4a,5,8,8a-tetrahydro-2H-phthalazin-1-one,
(cis)-4-(3-Chloro-4-methoxyphenyl)-2-(1-oxo-hexahydro-1l⁴-thiopyranyl)-4a,5,8,8a-tetrahydro-2H-phthalazin-1-one,
(cis)-4-(3,4-Diethoxyphenyl)-2-(1,1-dioxohexahydro-1l⁸-thiopyranyl)-4a,5,8,8a-tetrahydro-2H-phthalazin-1-one,
(cis)-4-(2,3-Dihydro-2,2-dimethyl-7-methoxybenzofuran-4-yl)-2-(1,1-dioxohexahydro-1l⁶-thiopyran-4-yl)-4a,5,8,8a-tetrahydro-2H-phthalazin-1-one,
(4aR,8aS)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one,
(4aS,8aR)-(cis)-4-(3,4-Dimethoxyphenyl)-2-(1,1-dioxohexahydro-1l⁶-thiopyran-4-yl)-4a,5,8,8a-tetrahydro-2H-phthalazin-1-one and
(cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one
   or the pharmaceutically acceptable salts thereof.

Particularly preferred compounds of embodiment A in this connection are compounds of formula 1 selected from
(cis)-4-(2,3-Dihydro-2,2-dimethyl-7-methoxybenzofuran-4-yl)-2-(tetrahydrothiopyran-4-yl)-4a,5,8,8a-tetrahydro-2H-phthalazin-1-one,
(4aS,8aR)-(cis)-4-(3,4-Dimethoxyphenyl)-2-(1,1-dioxohexahydro-1l⁶-thiopyranyl)-4a,5,8,8a-tetrahydro-2H-phthalazin-1-one and
(cis)-4-(3-Cyclopentyloxy-4-methoxyphenyl)-2-(1,1-dioxohexahydro-1l⁶-thiopyran-4-yl)-4a,5,8,8a-tetrahydro-2H-phthalazin-1-one
   or the pharmaceutically acceptable salts thereof.

The preparation of the compounds of embodiment A as well as their use as PDE4 inhibitors is disclosed in the International Patent application WO01/30777.

Another class of PDE4 inhibitors that may be usefully employed in the present invention includes compounds of formula 2 (Embodiment B) 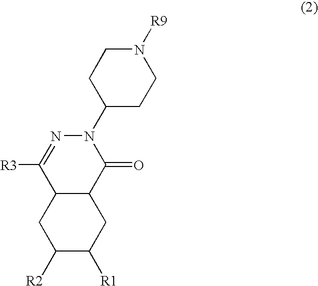
in which
R1 and R2 are both hydrogen or together form an additional bond,
R3 represents a benzene derivative of formula (a) or (b) 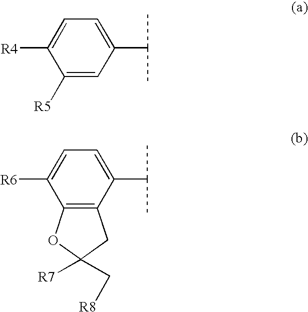
   wherein
   R4 is 1-4C-alkoxy or 1-4C-alkoxy which is completely or predominantly substituted by fluorine,
   R5 is 1-8C-alkoxy, 3-7C-cycloalkoxy, 3-7C-cycloalkylmethoxy, or 1-4C-alkoxy which is completely or predominantly substituted by fluorine,
   R6 is 1-4C-alkoxy, 3-5C-cycloalkoxy, 3-5C-cycloalkylmethoxy, or 1-4C-alkoxy which is completely or predominantly substituted by fluorine,
   R7 is 1-4AC-alkyl and
   R8 is hydrogen or 1-4C-alkyl,
   or wherein
   R7 and R8 together and with inclusion of the two carbon atoms, to which they are bonded, form a spiro-linked 5-, 6- or 7-membered hydrocarbon ring, optionally interrupted by an oxygen or sulphur atom,
R9 is 1-4C-alkyl, —S(O)₂—R10, —S(O)₂—(CH₂)ₙ—R11, —(CH₂)ₘ—S(O)₂—R12, —C(O)R13, —C(O)—(CH₂)ₙ—R14, —(CH₂)ₘ—C(O)—R15, Hetaryl, Aryl1 or 1-4C-alkyl-Aryl2,
R10 is 1-4C-alkyl, 5-dimethylaminonaphthalin-1-yl, —N(R16)R17, phenyl or phenyl substituted by R18 and/or R19,
R11 is —N(R16)R17,
R12 is —N(R16)R17,
R13 is 1-4C-alkyl, hydroxycarbonyl-1-4C-alkyl, phenyl, pyridyl, 4-ethyl-piperazin-2,3-dion-1-yl or —N(R16)R17,
R14 is —N(R16)R17,
R15 is —N(R16)R17, phenyl, phenyl substituted by R18 and/or R19 and/or R20,
R16 and R17 are independent from each other hydrogen, 1-7C-alkyl, 3-7C-cycloalkyl, 3-7C-cycloalkylmethyl, phenyl or phenyl substituted by R18 and/or R19 and/or R20, or R16 and R17 together and with inclusion of the nitrogen atom to which they are bonded, form a 4-morpholinyl-, 1-pyrrolidinyl-, 1-piperidinyl-, 1-hexahydroazepino- or a 1-piperazinyl-ring of formula (c) 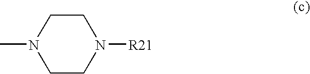
   wherein
   R21 is pyrid-4-yl, pyrid-4-ylmethyl, 1-4C-alkyldimethylamino, dimethylaminocarbonylmethyl, N-methyl-piperidin-4-yl, 4-morpholino-ethyl or tetrahydrofuran-2-ylmethyl,
R18 is halogen, nitro, cyano, carboxyl, 1-4C-alkyl, trifluoromethyl, 1-4C-alkoxy, 1-4C-alkoxycarbonyl, amino, mono- or di-1-4C-alkylamino, aminocarbonyl 1-4C-alkylcarbonylamino or mono- or di-1-4C-alkylaminocarbonyl,
R19 is halogen, amino, nitro, 1-4C-alkyl or 1-4C-alkoxy,
R20 is halogen,
Hetaryl is pyrimidin-2-yl, thieno-[2,3-d]pyrimidin-4-yl, 1-methyl-1H-pyrazolo-[3,4d]pyrimidin-4-yl, thiazolyl, imidazolyl or furanyl,
Aryl1 is pyridyl, phenyl or phenyl substituted by R18 and/or R19,
Aryl2 is pyridyl, phenyl, phenyl substituted by R18 and/or R19, 2-oxo-2H-chromen-7-yl or 4-(1,2,3-thiadiazol-4-yl)phenyl,
n is an integer from 1 to 4,
m is an integer from 1 to 4,
   or the pharmaceutical acceptable salts thereof.

Compounds of embodiment B which are to be emphasized in this connection are those compounds of formula 2 in which
R1 and R2 together form an additional bond,
R3 represents a benzene derivative of formula (a) or (b) 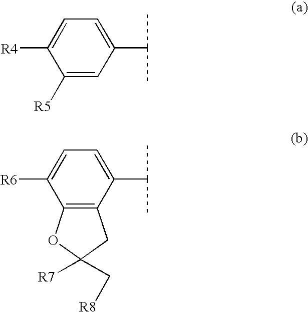
   wherein
   R4 is methoxy or ethoxy,
   R5 is methoxy or ethoxy,
   R6 is methoxy or ethoxy,
   R7 is methyl and
   R8 is hydrogen,
R9 is toluene-4-sulfonyl, methanesulfonyl, acetyl, 5-oxo-pentanoic acid, pyridin-4-yl-carbonyl, tert-butylaminocarbonyl, phenylaminocarbonyl, 5-dimethylamino-naphthalene-1-sulfonyl, 4-nitro-phenyl, pyridin-4-ylmethyl, morpholine-4-carbonyl, 2-(4-amino-3,5-dichlorophenyl)-2-oxo-ethyl, 1-methyl-1H-pyrazolo[3,4-d]pyrimidin-4-yl, thieno[2,3-d]pyrimidin-4-yl, pyrimidin-2-yl, 2-oxo-2H-chromen-7-ylmethyl, isopropyl, morpholin-4-yl-2-oxo-ethyl, phenethyl, pyridin-3-ylmethyl, pyridin-2-ylmethyl, pyridin-4-ylmethyl, 2-morpholin-4-ylethanoyl, 2-[4-(2-dimethylamino-ethyl)-piperazin-1-yl]-ethanoyl, isopropylaminocarbonylmethyl, 4-ethyl-piperazine-2,3-dione-1-carbonyl, 4-(1,2,3-thiadiazol-4-yl-)benzyl, 4-ethoxycarbonylphenylamino-2-oxo-ethyl or aminocarbonyl-methyl,
   or the pharmaceutical acceptable salts thereof.

Preferred compounds of embodiment B in this connection are compounds of formula 2 selected from
(4aS,8aR)-4-(3,4-Diethoxyphenyl)-2-[1-(toluene-4-sulfonyl)-piperidin-4-yl]4a,5,8,8a-tetrahydro-2H-phthalazin-1-one,
(4aS,8aR)-4-(3,4-Diethoxyphenyl)-2-(1-methanesulfonyl-piperidin-4-yl)-4a,5,8,8a-tetrahydro-2H-phthalazin-1-one,
(4aS,8aR)-2-(1-Acetyl-piperidin-4-yl)-4-(3,4-diethoxyphenyl)-4a,5,8,8a-tetrahydro-2H-phthalazin-1-one,
5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid,
(4aS,8aR)-4-(3,4-Diethoxyphenyl)-2-[1-(1-pyridin-4-yl-methanoyl)-piperidin-4-yl]4a,5,8,8a-tetrahydro-2H-phthalazin-1-one,
4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide,
4-[(4aS,8aR)-4-(3,4-Diethoxyphenyl)-1-oxo-4a,5,8,8a-tetrahydro-1H-phthalazin-2-yl]-piperidine-1-carboxylic acid phenylamide,
4-[(4aS,8aR)-4-(3,4-Dimethoxyphenyl)-1-oxo-4a,5,8,8a-tetrahydro-1H-phthalazin-2-yl]-piperidine-1-carboxylic acid tert-butylamide,
(cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide,
(4aS,8aR)-4-(3,4-Dimethoxyphenyl)-2-[1-(5-dimethylamino-naphthalene-1-sulfonyl)piperidin-4-yl]-4a,5,8,8a-tetrahydro-2H-phthalazin-1-one,
(4aS,8aR)-4-(3,4-Dimethoxyphenyl)-2-[1-(4-nitro-phenyl)-piperidin-4-yl]-4a,5,8,8a-tetrahydro-2H-phthalazin-1-one,
(4aS,8aR)-4-(3,4-Dimethoxyphenyl)-2-(1-pyridin-4-ylmethyl-piperidin-4-yl)-4a,5,8,8a-tetrahydro-2H-phthalazin-1-one,
(4aS,8aR)-4-(3,4-Dimethoxyphenyl)-2-[1-(morpholin-4-carbonyl)-piperidin-4-yl]-4a,5,8,8a-tetrahydro-2H-phthalazin-1-one,
(4aS,8aR)-2-{1-[2-(4-Amino-3,5-dichloro-phenyl)-2-oxoethyl]-piperidin-4-yl}-4-(3,4-dimethoxy-phenyl)-4a,5,8,8a-tetrahydro-2H-phthalazin-1-one,
4-(3,4-Dimethoxyphenyl)-2-[1-(1-methyl-1H-pyrazolo[3,4]pyrimidin-4-yl)piperidin-4-yl]4a,5,8,8a-tetrahydro-2H-naphthalen-1-one,
(4aS,8aR)-4-(3,4-Dimethoxyphenyl)-2-(1-thieno[2,3]pyrimidin-4-yl-piperidin-4-yl)-4a,5,8,8a-tetrahydro-2H-phthalazin-1-one,
(4aS,8aR)-4-(3,4-Dimethoxyphenyl)-2-(1-pyrimidin-2-yl-piperidin-4-yl)-4a,5,8,8a-tetrahydro-2H-phthalazin-1-one,
(4aS,8aR)-4-(3,4-Dimethoxyphenyl)-2-[1-(2-oxo-2H-chromen-7-ylmethyl)piperidin-4-yl]-4a,5,8,8a-tetrahydro-2H-phthalazin-1-one,
4-(3,4-Dimethoxyphenyl)-2-(1-isopropyl-piperidin-4-yl)-4a,5,8,8a-tetrahydro-2H-phthalazin-1-one,
(4aS,8aR)-4-(3,4-Dimethoxyphenyl)-2-[1-(2-morpholin-4-yl-2-oxo-ethyl)-piperidin-4-yl]-4a,5,8,8a-tetrahydro-2H-phthalazin-1-one,
(4aS,8aR)-4-(3,4-Dimethoxyphenyl)-2-(1-phenethyl-piperidin-4-yl)-4a,5,8,8a-tetrahydro-2H-phthalazin-1-one,
(4aS,8aR)-4-(3,4-Diethoxyphenyl)-2-[1-(morpholine-4-carbonyl)-piperidin-4-yl]-4a,5,8,8a-tetrahydro-2H-phthalazin-1-one,
(4aS,8aR)-4-(3,4-Dimethoxyphenyl)-2-(1-pyridin-3-ylmethyl-piperidin-4-yl)-4a,5,8,8a-tetrahydro-2H-phthalazin-1-one,
(4aS,8aR)-4-(3,4-Dimethoxy-phenyl)-2-(1-pyridin-2-ylmethyl-piperidin-4-yl)-4a,5,8,8a-tetrahydro-2H-phthalazin-1-one,
(4aS,8aR)-4-(3,4-Diethoxyphenyl)-2-[1-(2-morpholin-4-yl-ethanoyl)-piperidin-4-yl]4a,5,8,8a-tetrahydro-2H-phthalazin-1-one,
(4aS,8aR)-4-(3,4-Diethoxyphenyl)-2-(1-{2-[4-(2-dimethylamino-ethyl)-piperazin-1-yl]ethanoyl}-piperidin-4-yl)-4a,5,8,8a-tetrahydro-2H-phthalazin-1-one,
2-{4-[(4aS,8aR)-4-(3,4-Dimethoxyphenyl)-1-oxo-4a,5,8,8a-tetrahydro-1H-phthalazin-2-yl]piperidin-1-yl}-N-isopropyl-acetamide,
(4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one,
1-(1-{4-[(4aS,8aR)-4-(3,4-Dimethoxyphenyl)-1-oxo-4a,5,8,8a-tetrahydro-1H-phthalazin-2-yl]-piperidin-1-yl}-methanoyl)-4-ethylpiperazine-2,3-dione,
4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)benzoic acid ethyl ester and
2-{4-[(4aS,8aR)-4-(3,4-Dimethoxyphenyl)-1-oxo-4a,5,8,8a-tetrahydro-1H-phthalazin-2-yl]piperidin-1-yl}-acetamide,
   or the pharmaceutical acceptable salts thereof.

Further preferred compounds of embodiment B in this connection are compounds of formula 2 selected from
(4aS,8aR)-4-(3,4-Diethoxyphenyl)-2-[1-(toluenesulfonyl)piperidin-4-yl]-4a,5,8,8a-tetrahydro-2H-phthalazin-1-one,
(4aS,8aR)-4-(3,4-Diethoxyphenyl)-2-(1-methanesulfonyl-piperidin-4-yl)-4a,5,8,8a-tetrahydro-2H-phthalazin-1-one,
(4aS,8aR)-2-(1-Acetyl-piperidin-4-yl)-4-(3,4-diethoxyphenyl)-4a,5,8,8a-tetrahydro-2H-phthalazin-1-one,
5-{4-[(4aS,8aR)-4-(3,4-Diethoxy-phenyl)-1-oxo-4a,5,8,8a-tetrahydro-1H-phthalazin-2-yl]-piperidin-1-yl}-5-oxo-pentanoic acid,
(4aS,8aR)-4-(3,4-Diethoxyphenyl)-2-[1-(1-pyridin-4-yl-methanoyl)-piperidin-4-yl]-4a,5,8,8a-tetrahydro-2H-phthalazin-1-one,
4-[(4aS,8aR)-4-(3,4-Diethoxyphenyl)-1-oxo-4a,5,8,8a-tetrahydro-1H-phthalazin-2-yl]-piperidine-1-carboxylic acid tert-butylamide,
4-[(4aS,8aR)-4-(3,4-Diethoxyphenyl)-1-oxo-4a,5,8,8a-tetrahydro-1H-phthalazin-2-yl]piperidine-1-carboxylic acid phenylamide,
(cis)-4-[4-(7-Methoxy-2,2-dimethyl-2,3-dihydro-benzofuran-4-yl)-1-oxo-4a,5,8,8a-tetrahydro-1H-phthalazin-2-yl]-piperidine-1-carboxylic acid tert-butylamide,
(4aS,8aR)-4-(3,4-Dimethoxyphenyl)-2-[1-(5-dimethylamino-naphthalene-1-sulfonyl)-piperidin-4-yl]-4a,5,8,8a-tetrahydro-2H-phthalazin-1-one,
(4aS,8aR)-4-(3,4-Dimethoxyphenyl)-2-[1-(4-nitro-phenyl)-piperidin-4-yl]-4a,5,8,8a-tetrahydro-2H-phthalazin-1-one,
(4aS,8aR)-4-(3,4-Dimethoxyphenyl)-2-(1-pyridin-4-ylmethyl-piperidin-4-yl)-4a,5,8,8a-tetrahydro-2H-phthalazin-1-one,
(4aS,8aR)-4-(3,4-Dimethoxyphenyl)-2-[1-(morpholine-4-carbonyl)-piperidin-4-yl]-4a,5,8,8a-tetrahydro-2H-phthalazin-1-one,
(4aS,8aR)-2-{1-[2-(4-Amino-3,5-dichloro-phenyl)-2-oxo-ethyl]-piperidin-4-yl}-4-(3,4-dimethoxy-phenyl)-4a,5,8,8a-tetrahydro-2H-phthalazin-1-one,
4-(3,4-Dimethoxyphenyl)-2-[1-(1-methyl-1H-pyrazolo[3,4-d]pyrimidin-4-yl)piperidin-4-yl]4a,5,8,8a-tetrahydro-2H-naphthalen-1-one,
(4aS,8aR)-4-(3,4-Dimethoxyphenyl)-2-(1-thieno[2,3-d]pyrimidin-4-yl-piperidin-4-yl)-4a,5,8,8a-tetrahydro-2H-phthalazin-1-one,
(4aS,8aR)-4-(3,4-Dimethoxyphenyl)-2-(1-pyrimidin-2-yl-piperidin-4-yl)-4a,5,8,8a-tetrahydro-2H-phthalazin-1-one,
(4aS,8aR)-4-(3,4-Dimethoxyphenyl)-2-[1-(2-oxo-2H-chromen-7-ylmethyl)-piperidin-4-yl]-4a,5,8,8a-tetrahydro-2H-phthalazin-1-one,
4-(3,4-Dimethoxyphenyl)-2-(1-isopropyl-piperidin-4-yl)-4a,5,8,8a-tetrahydro-2H-phthalazin-1-one,
(4aS,8aR)-4-(3,4-Dimethoxyphenyl)-2-[1-(2-morpholin-4-yl-2-oxo-ethyl)-piperidin-4-yl]4a,5,8,8a-tetrahydro-2H-phthalazin-1-one,
(4aS,8aR)-4-(3,4-Dimethoxyphenyl)-2-(1-phenethyl-piperidin-4-yl)-4a,5,8,8a-tetrahydro-2H-phthalazin-1-one,
(4aS,8aR)-4-(3,4-Diethoxyphenyl)-2-[1-(morpholine-4-carbonyl)-piperidin-4-yl]4a,5,8,8a-tetrahydro-2H-phthalazin-1-one, (4aS,8aR)-4-(3,4-Dimethoxyphenyl)-2-(1-pyridin-3-ylmethyl-piperidin-4-yl)-4a,5,8,8a-tetrahydro-2H-phthalazin-1-one,
(4aS,8aR)-4-(3,4-Dimethoxy-phenyl)-2-(1-pyridin-2-ylmethyl-piperidin-4-yl)-4a,5,8,8a-tetrahydro-2H-phthalazin-1-one,
(4aS,8aR)-4-(3,4-Diethoxyphenyl)-2-[1-(2-morpholin-4-yl-ethanoyl)-piperidin-4-yl]-4a,5,8,8a-tetrahydro-2H-phthalazin-1-one,
(4aS,8aR)-4-(3,4-Diethoxyphenyl)-2-(1-{2-[4-(2-dimethylamino-ethyl)-piperazin-1-yl]-ethanoyl}-piperidin-4-yl)-4a,5,8,8a-tetrahydro-2H-phthalazin-1-one,
2-{4-[(4aS,8aR)-4-(3,4-Dimethoxyphenyl)-1-oxo-4a,5,8,8a-tetrahydro-1H-phthalazin-2-yl]-piperidin-1-yl}-isopropyl-acetamide,
(4aS,8aR)-4-(3,4-Dimethoxyphenyl)-2-[1-(4-1,2,3-thiadiazol-4-yl-benzyl)-piperidin-4-yl]-4a,5,8,8a-tetrahydro-2H-phthalazin-1-one,

1-(1-{4-[(4aS,8aR)-4-(3,4-Dimethoxyphenyl)-1-oxo-4a,5,8,8a-tetrahydro-1H-phthalazin-2-yl]piperidin-1-yl}-methanoyl)-4-ethyl-piperazine-2,3-dione,
4-(2-{4-[(4aS,8aR)-4-(3,4-Dimethoxy-phenyl)-1-oxo-4a,5,8,8a-tetrahydro-1H-phthalazin-2-yl]-piperidin-1-yl}-ethanoylamino)-benzoic acid ethyl ester and
2-{4-[(4aS,8aR)-4-(3,4-Dimethoxyphenyl)-1-oxo-4a,5,8,8a-tetrahydro-1H-phthalazin-2-yl]-piperidin-1-yl}-acetamide,
   or the pharmaceutically acceptable salts thereof.

Particularly preferred compounds of embodiment B in this connection are compounds of formula 2 selected from
(4aS,8aR)-4-(3,4-Dimethoxyphenyl)-2-(1-pyrimidin-2-yl-piperidin-4-yl)-4a,5,8,8a-tetrahydro-2H-phthalazin-1-one,
(4aS,8aR)-4-(3,4-Dimethoxy-phenyl)-2-(1-pyridin-2-ylmethyl-piperidin-4-yl)-4a,5,8,8a-tetrahydro-2H-phthalazin-1-one,
2-{4-[(4aS,8aR)-4-(3,4-Dimethoxyphenyl)-1-oxo-4a,5,8,8a-tetrahydro-1H-phthalazin-2-yl]-piperidin-1-yl}-acetamide,
   or the pharmaceutically acceptable salts thereof.

The preparation of the compounds of embodiment B as well as their use as PDE4 inhibitors is disclosed in the International Patent application WO02/064584.

Still another group of PDE4 inhibitors (embodiment C) that may be usefully employed in the present invention includes the following compounds:
N-(3,5-dichloropyrid-4-yl)-3-cyclopentyloxy 4-methoxybenzamide [INN: PICLAMILAST] and its salts; the preparation of this compound and its pharmaceutically acceptable salts as well as their use as PDE4 inhibitors is disclosed in the international patent application WO92/12961
3-cyclopropylmethoxy-4-difluoromethoxy-N-(3,5-dichloropyrid-4-yl)-benzamide [INN: ROFLUMILAST] and its salts; the preparation of this compound and its pharmaceutically acceptable salts as well as their use as PDE4 inhibitors is disclosed in the international patent application WO95/01338
3-cyclopropylmethoxy-4-difluoromethoxy-N-(3,5-dichloro-1-oxy-pyridyl)-benzamide (Roflumilast-N-Oxide) and its salts; the preparation of this compound and its pharmaceutically acceptable salts as well as their use as PDE4 inhibitors is disclosed in the international patent application WO95/01338
3-[3-(cyclopentyloxy)-4-methoxybenzyl]-6-(ethylamino)-8-isopropyl-3H-purine [Research Code: V-11294A]; the preparation of this compound and its pharmaceutically acceptable salts as well as their use as PDE4 inhibitors is disclosed in the international patent application WO95/00516
N-[9-methyl-4-oxo-1-phenyl-3,4,6,7-tetrahydropyrrolo[3,2,1-jk][1,4]benzo-diazepin-3(R)-yl]pyridine-4-carboxamide [Research Code: CI-1018]; the preparation of this compound and its pharmaceutically acceptable salts as well as their use as PDE4 inhibitors is disclosed in the international patent application WO96/11690.
3,7-dihydro-3-(4-chlorophenyl)-1-propyl-1H-purine-2,6-dione [INN AROFYLLINE]; the preparation of this compound and its pharmaceutically acceptable salts as well as their use as PDE4 inhibitors is disclosed in the European patent application EP0435811.
N-(3,5-dichloro-4-pyridinyl)-2-[1-(4-fluorobenzyl)-5-hydroxy-1H-indol-3-yl]-2-oxoacetamide [Research Code: AWD-12-281]; the preparation of this compound and its pharmaceutically acceptable salts as well as their use as PDE4 inhibitors is disclosed in the international patent application WO98/09946
N-(3,5-dichloropyridin-4-yl)-2-[5-fluoro-1-(4-fluorobenzyl)-1H-indo-3-yl-2-oxoacetamide [Research Code: AWD-12-343]; the preparation of this compound and its pharmaceutically acceptable salts as well as their use as PDE4 inhibitors is disclosed in the international patent application WO98/09946.
Tetrahydro-5-[4-methoxy-3-[(1S,2S,4R)-2-norbornyloxy]phenyl]-2(1H)-pyrimidone [INN: ATIZORAM]; the preparation of this compound and its pharmaceutically acceptable salts as well as their use as PDE4 inhibitors is disclosed in the European patent application EP0389282.
β-[3-(cyclopentyloxy)-4-methoxyphenyl]-1,3-dihydro-1,3-dioxo-2H-isoindole-2-propanamide [Research Code: CDC-801]; the preparation of this compound and its pharmaceutically acceptable salts as well as their use as PDE4 inhibitors is disclosed in the international patent application WO97/23457.
Methanesulfonic acid 2-(2,4-dichlorophenylcarbonyl)-3-ureidobenzo-furanyl-6-yl ester [INN: LIRIMILAST]; the preparation of this compound and its pharmaceutically acceptable salts as well as their use as PDE4 inhibitors is disclosed in the European patent application EP0731099.
3,5-dichloro-4-[8-methoxy-2-(trifluoromethyl)quinolin-5-ylcarbox-amido]pyridine-1-oxide (Research Code: SCH-351591]; the preparation of this compound and its pharmaceutically acceptable salts as well as their use as PDE4 inhibitors is disclosed in the international patent application WO00/26208;
cis-4-cyan-4-[3-cyclopentyloxy-4-methoxyphenyl]cyclohexane-1-carboxylic acid [INN: Cilomilast], the preparation of this compound and its pharmaceutically acceptable salts as well as their use as PDE4 inhibitors is disclosed in the International patent application WO93/19749
   as well as the compounds with the research codes CDC-998, D4396, IC-485, CC-1088 and KW4490 and their pharmaceutically acceptable salts.

Preferred compounds of embodiment C are in this connection
N-(3,5-dichloro-4-pyridinyl)-2-[1-(4-fluorobenzyl)-5-hydroxy-1H-indol-3-yl]-2-oxoacetamide [Research Code: AWD12-281],
cis-4-cyano-4-[3-cyclopentyloxy-4-methoxyphenyl]cyclohexane-1-carboxylic acid [INN: Cilomilast],
3-cyclopropylmethoxy-4-difluoromethoxy-N-(3,5-dichloropyrid-4-yl)-benzamide [INN: ROFLUMILAST],
3-cyclopropylmethoxy-4-difluoromethoxy-N-(3,5-dichloro-1-oxy-pyrid-4-yl)-benzamide (Roflumilast-N-Oxide) and their pharmaceutically acceptable salts.

Particularly preferred compounds of embodiment C are in this connection
3-cyclopropylmethoxy-4-difluoromethoxy-N-(3,5-dichloropyrid-4-yl)-benzamide [INN: ROFLUMILAST],
3-cyclopropylmethoxy-4-difluoromethoxy-N-(3,5-dichloro-1-oxy-pyrid-4-yl)-benzamide (Roflumilast-N-Oxide) and their pharmaceutically acceptable salts.

1-4C-Alkyl is a straight-chain or branched alkyl radical having 1 to 4 carbon atoms. Examples are the butyl, isobutyl, sec-butyl, tert-butyl, propyl, isopropyl, ethyl and methyl radicals.

1-4C-Alkoxy is a radical which, in addition to the oxygen atom, contains a straight-chain or branched alkyl radical having 1 to 4 carbon atoms. Alkoxy radicals having 1 to 4 carbon atoms, which may be mentioned in this context are, for example, the butoxy, isobutoxy, sec-butoxy, tert-butoxy, propoxy, isopropoxy, ethoxy and methoxy radicals.

1-8C-Alkoxy is a radical which, in addition to the oxygen atom, contains a straight-chain or branched alkyl radical having 1 to 8 carbon atoms. Alkoxy radicals having 1 to 8 carbon atoms which may be mentioned in this context are, for example, the octyloxy, heptyloxy, isoheptyloxy (5-methylhexyloxy), hexyloxy, isohexyloxy (4-methylpentyloxy), neohexyloxy (3,3-dimethylbutoxy), pentyloxy, isopentyloxy (3-methylbutoxy), neopentyloxy (2,2-dimethylpropoxy), butoxy, isobutoxy, sec-butoxy, tert-butoxy, propoxy, isopropoxy, ethoxy and methoxy radicals.

Halogen within the meaning of the present invention is bromine, chlorine or fluorine.

3-7C-Cycloalkoxy stands for cyclopropyloxy, cyclobutyloxy, cyclopentyloxy, cyclohexyloxy or cycloheptyloxy, of which cyclopropyloxy, cyclobutyloxy and cyclopentyloxy are preferred.

3-7C-Cycloalkylmethoxy stands for cyclopropylmethoxy, cyclobutylmethoxy, cyclopentylmethoxy, cyclohexylmethoxy or cycloheptylmethoxy, of which cyclopropylmethoxy, cyclobutylmethoxy and cyclopentylmethoxy are preferred.

3-5C-Cycloalkoxy stands for cyclopropyloxy, cyclobutyloxy and cyclopentyloxy.

3-5C-Cycloalkylmethoxy stands for cyclopropylmethoxy, cyclobutylmethoxy and cyclopentylmethoxy.

1-4C-Alkoxy which is completely or predominantly substituted by fluorine is, for example, the 2,2,3,3,3-pentafluoropropoxy, the perfluoroethoxy, the 1,2,2-trifluoroethoxy and in particular the 1,1,2,2-tetrafluoroethoxy, the 2,2,2-trifluoroethoxy, the trifluoromethoxy and the difluoromethoxy radical, of which the difluoromethoxy radical is preferred. “Predominantly” in this connection means that more than half of the hydrogen atoms of the 1-4C-alkoxy group are replaced by fluorine atoms.

As spiro-linked 5-, 6- or 7-membered hydrocarbon rings, optionally interrupted by an oxygen or sulphur atom, may be mentioned the cyclopentane, cyclohexane, cycloheptane, tetrahydrofuran, tetrahydropyran and the tetrahydrothiophen ring.

1-4C-Alkylcarbonyl is a carbonyl group to which one of the abovementioned 1-4C-alkyl radicals is bonded. An example is the acetyl radical [CH₃C(O)—].

An 1-4C-Alkylcarbonylamino radical is, for example, the propionylamino [C₃H₇C(O)NH—] and the acetylamino radical [CH₃C(O)NH—].

Mono- or Di-1-4C-alkylamino radicals contain in addition to the nitrogen atom, one or two of the abovementioned 1-4C-alkyl radicals. Preferred are the di-1-4C-alkylamino radicals, especially the dimethylamino, the diethylamino and the diisopropylamino radical.

Mono- or Di-1-4C-alkylaminocarbonyl radicals contain in addition to the carbonyl group one of the abovementioned mono- or di-1-4C-alkylamino radicals. Examples which may be mentioned are the N-methyl- the N,N-dimethyl-, the N-ethyl-, the N-propyl-, the N,N-diethyl- and the N-isopropylaminocarbonyl radical.

Salts encompassed within the term “pharmaceutically acceptable salts” refer to non-toxic salts of the compounds of this invention which are generally prepared by reacting a free base with a suitable organic or inorganic acid or by reacting the acid with a suitable organic or inorganic base. Particular mention may be made of the pharmaceutically acceptable inorganic and organic acids customarily used in pharmacy. Those suitable are water-soluble and water-insoluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in salt preparation—depending on whether a mono- or polybasic acid is concerned and depending on which salt is desired—in an equimolar quantitative ratio or one differing therefrom.

For the purposes of this invention the expression “neoplasms of lymphoid cells” includes leukemia, lymphoma and myeloma. More specifically it includes the different types of leukemia, the myelodysplastic syndromes and lymphoma.

The expression “different types of leukemia” includes the myeloid leukemias CML (chronic myeloid leukemia), AML (acute myeloid leukemia) and ANLL (acute nonlymphocytic leukemia) as well as the lymphocytic leukemias ALL (acute lymphocytic leukemia), CLL (chronic lymphocytic leukemia) and HCL (hairy cell leukemia). AML can further be subclassified in acute promyelocytic leukemia (APL), acute myelomonocytic leukemia, acute monocytic leukemia, acute erythroleukemia and acute megakaryocytic leukemia. APL is a rare form of acute myelogenous leukemia with typical chromosomal translocations leading to the expression of abnormal fusion proteins involving the nuclear retinoic acid receptor, RARα.

The myeloplastic syndromes (MDS) are heterogenous clonal hematopoietic stem cell disorders grouped together because of the presence of dysplastic changes in one or more of the hematopoietic lineages. MDS were previously referred to as smoldering leukemia or preleukeamia, oligoblastic leukemia or hematopoietic dysplasia, implying an indolent course.

Diffuse large B cell lymphoma (DLBCL) is the most common lymphoid malignancy in adults; it is curable only in less than 50% of patients. Lymphomas are typically subdivided into Hodkin's- and non-Hodkin's lymphoma.

In a first aspect of the present invention, there is provided the use of a compound (compound to be emphasized, preferred compound, particularly preferred compound) of embodiment A, B or C in the preparation of a pharmaceutical composition for the treatment of neoplasms of lymphoid cells.

In a second aspect of the present invention there is provided a method of treating neoplasms of lymphoid cells in a mammal including administering to the mammal a therapeutically effective amount of a compound (compound to be emphasized, preferred compound, particularly preferred compound) of embodiment A, B or C.

In a third aspect of the present invention, there is provided a treatment combination for neoplasms of lymphoid cells, including: therapeutically effective amounts of (i) a compound (compound to be emphasized, preferred compound, particularly preferred compound) of embodiment A, B or C; and (ii) one or more differentiation inducing agents and/or an agent effective in raising intracellular concentrations of cAMP or a stable analogue of cAMP.

In a forth aspect of the present invention, there is provided a treatment combination for neoplasms of lymphoid cells, including: therapeutically effective amounts of (i) a compound (compound to be emphasized, preferred compound, particularly preferred compound) of embodiment A, B or C; and (ii) one or more differentiation inducing agents.

In a fifth aspect of the present invention, there is provided a treatment combination for neoplasms of lymphoid cells, including: therapeutically effective amounts of (i) a compound (compound to be emphasized, preferred compound, particularly preferred compound) of embodiment A, B or C; and (ii) an agent effective in raising intracellular concentrations of cAMP or a stable analogue of cAMP.

In a sixth aspect of the present invention, there is provided the use of a compound (compound to be emphasized, preferred compound, particularly preferred compound) of embodiment A, B or C and one or more differentiation inducing agents and/or an agent effective in raising intracellular concentrations of cAMP or a stable analogue of cAMP in the preparation of a pharmaceutical composition for the treatment of neoplasms of lymphoid cells.

In a seventh aspect of the present invention, there is provided the use of a compound (compound to be emphasized, preferred compound, particularly preferred compound) of embodiment A, B or C and one or more differentiation inducing agents in the preparation of a pharmaceutical composition for the treatment of neoplasms of lymphoid cells.

In a eighth aspect of the present invention, there is provided the use of a compound (compound to be emphasized, preferred compound, particularly preferred compound) of embodiment A, B or C and an agent effective in raising intracellular concentrations of cAMP or a stable analogue of cAMP in the preparation of a pharmaceutical composition for the treatment of neoplasms of lymphoid cells.

In a ninth aspect of the present invention, there is provided a method of treating neoplasms of lymphoid cells in a mammal, including: administering to said mammal therapeutically effective amounts of (i) a compound (compound to be emphasized, preferred compound, particularly preferred compound) of embodiment A, B or C; and (ii) one or more differentiation inducing agents and/or an agent effective in raising intracellular concentrations of cAMP or a stable analogue of cAMP.

In a tenth aspect of the present invention, there is provided a method of treating neoplasms of lymphoid cells in a mammal, including: administering to said mammal therapeutically effective amounts of (i) a compound (compound to be emphasized, preferred compound, particularly preferred compound) of embodiment A, B or C; and (ii) one or more differentiation inducing agents.

In a twelfth aspect of the present invention, there is provided a method of treating neoplasms of lymphoid cells in a mammal, including: administering to said mammal therapeutically effective amounts of (i) a compound (compound to be emphasized, preferred compound, particularly preferred compound) of embodiment A, B or C; and (ii) an agent effective in raising intracellular concentrations of cAMP or a stable analogue of cAMP.

As recited above, in one aspect of the present invention a method of treating neoplasms of lymphoid cells is provided, which includes administering therapeutically effective amounts of (i) a compound of embodiment A, B or C; and (ii) one or more differentiation inducing agents and/or an agent effective in raising intracellular concentrations of cAMP or a stable analogue of cAMP.

Typical differentiation inducing agents useful in the present invention include, but are not limited to, ATRA (all trans retinoic acid), 13-cis-retinoic acid, CD437 [6-(3-(1-adamantyl)-4-hydroxyphenyl)-2-naphthalene carboxylic acid], rexinoids (e.g. LG1069, LG100268, bexarotene, CD2809), HDAC inhibitors [histone deacetylase inhibitors, e.g. N-[4-[N-(2-aminophenyl)carbamoyl]benzyl]carbamic acid 3-pyridylmethyl ester (Research Code: MS-27-275, EP 0847992); N-hydroxy-N′-phenyloctanediamide (Research Code: SAHA; WO93/07148); 4-acetamido-N-(2-aminophenyl)benzamide (Research Code: PD-123654; EP 0242851); butanoic acid pivaloyloxymethyl ester (Research Code: AN-9; EP0302349); N′-hydroxy-N-(3-pyridyl)octane-1,8-dicarboxamide (INN: PYROXAMIDE); 3-[4-[N-(2-hydroxyethyl)-N-[2-(1H-indol-3-yl)ethyl]aminomethyl]phenyl]-2-propenohydroxamic acid (INN: DACINOSTAT; WO02/22577); N-[5-(N-hydroxycarbamoyl)pentyl]indane-2-carboxamide (Research Code: PX-117735; WO02/30879); 6-[2-(9H-fluoren-9-ylidene)acetamido]hexanohydroxamic acid (Research Code: PX-117456; WO02/26696); N-[5-(N-hydroxycarbamoyl)pentyl]naphthalene-2-carboxamide (Research Code: PX-117445; WO02/30879)], DNA methyltransferase inhibitors (e.g. 5-azacytidine), hematopoietic growth factors (e.g. G-CSF, GM-CSF), interferon α, interleukin 1, TRAIL, HMBA (hexamethylene bisacetamide), vitamin D3 and analogs (e.g. cholecalciferol), arsenic trioxide (Trisenox, Cell Therapeutics, Inc. Seattle, Wash.), EGCG (green tea catechin epigallocatechin-3-gallate), DNA topoisomerase II inhibitors (e.g. ICRF-154, ICRF-193, etoposide), taraxinic acid, verticinone, PPAR-gamma agonists (e.g. thiazolidinediones (TZDs), troglitazone), antibodies versus CD19, CD20 (rituximab), CD22 or CD52 (alemtuzumab), CD33-antibodies alone or as conjugate [e.g. mylotarg (CD33-calicheamicin)], alkylating cytostatika (e.g. cyclphosphamide, chlorambucil), purine analogs (thioguanine, fludarabine), cytosine-arabinosides (e.g. AraC), anticyclines (e.g. daunorubicine), vinca-alkaloids (e.g. vincristine) and glucocorticosteroids. Preferred are in this connection the histone deacetylase inhibitors and the all trans retinoic acid. Particularly preferred is the all trans retinoic acid.

As suitable agents effective in raising intracellular concentrations of cAMP may be mentioned agents which (1) increase cAMP levels by activating cell surface receptors which are Gs protein coupled to the cAMP generating enzyme adenylyl cyclase including, but not limited to, prostaglandin E2, prostacyclin derivatives (e.g. iloprost), dopamine, dobutamine, β2-adrenoreceptor agonists (for example: terbutaline, albuterol, pirbuterol, bitolterol, formoterol, salmeterol and salbutamol), adenosine A1 receptor agonists, and adenosine A2 receptor agonists; and (2) increase cAMP levels by directly stimulating adenylyl cyclase, including, but not limited to forskolin.

As examples of stable analogs of cAMP may be mentioned dibutyryl cAMP, 8-chloro-cAMP and 8-bromo cAMP.

The invention relates to several methods for the treatment of mammals, which are suffering from neoplasms of lymphoid cells. The term mammal includes the meaning human being.

The compound of embodiment A, B and C, the differentiation inducing agent(s) and/or the agent effective in raising intracellular concentrations of cAMP or the stable analogue of cAMP may be employed in combination in accordance with the invention by administration concomitantly in (1) a unitary pharmaceutical composition including both (or all three) active compounds or (2) in separate pharmaceutical compositions each including one of the active compounds. Alternatively, the active compounds of the combination may be administered separately in a sequential manner wherein the compound of embodiment A, B or C, the differentiation inducing agent(s), the agent effective in raising intracellular concentrations of cAMP or the stable analogue of cAMP is administered first and the other(s) second. Such sequential administration may be close in time or remote in time.

The compound of embodiment A, B or C, the differentiation inducing agent(s), the agents which are effective to raise intracellular cAMP concentrations and the stable analogs of cAMP of the present invention may be administered by any appropriate route. Suitable routes include oral, rectal, nasal, topical, parenteral (including subcutaneous, intramuscular, intravenous and intradermal) and by inhalation.

The treatment combinations and pharmaceutical compositions are prepared by processes, which are known per se and familiar to the person skilled in the art As treatment combinations or pharmaceutical compositions, the compounds the different compounds according to the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, aerosols, gels or solutions, the active compound(s) content advantageously being between 0.1 and 95% and where, by appropriate choice of the auxiliaries and/or excipients, a pharmaceutical administration form (e.g. a delayed release form or a enteric form) exactly suited to the active compound(s) and/or the desired onset of action can be achieved.

The person skilled in the art is familiar with auxiliaries or excipients, which are suitable for the desired pharmaceutical formulations on account of his/her expert knowledge. In addition to solvents, gel formers, ointment bases other active compound excipients, for example antioxidants, dispersants, emulsifiers, preservatives, solubilizers, colorants, complexing agents, or permeation promoters can be used.

As indicated, therapeutically effective amounts of the certain PDE4 inhibitors, and if utilized, the differentiation inducing agent(s) and/or the agent, that is effective to raise intracellular cAMP concentrations or the stable analogue of cAMP, are administered to the mammal.

It is known to the person skilled in the art that the optimal dose of an/the active compound(s) can vary as a function of the body weight, the age and the general condition of the patient, and his/her response behaviour to the active compound(s).

The customary dose of the PDE4 inhibitor compounds of embodiment A, B or C in the case of systemic therapy (p.o. or i.v.) is between 0.001 and 3 mg/kg body weight of recipient (mammal) per day.

In case of oral administration of 3-cyclopropylmethoxy-4-difluoromethoxy-N-(3,5-dichloropyrid-4-yl) benzamide (ROFLUMILAST), the adult daily dose is in the range from 50-1000 μg, preferably in the range from 250-500 μg, preferably by once daily administration.

The daily dosage of the differentiation inducing agent all trans retinoic acid (ATRA) is between 0.1 and 30 mg/kg body weight of recipient (mammal), and preferably from about 0.2 to about 5 mg/kg body weight of recipient (mammal). The daily dosage of the other indicated differentiation inducing agents may be from about 0.001 to about 100 mg/kg body weight of recipient, depending on the employed differentiation inducing agent.

The daily dosages of the agent, which is effective to raise intracellular cAMP concentrations may be from about 0.001 to about 15 mg/kg body weight of recipient (mammal).

Pharmacology

It was demonstrated in a study that N-(3,5-dichloropyrid-4-yl)-3-cyclopentyloxy-4-methoxybenzamide [INN: PICLAMILAST], a selective phosphodiesterase 4 (PDE4) inhibitor, potentiates the growth inhibitory and cyto-differentiating activities of all trans retinoic acid (ATRA) in NB4, HL-60 and U937 blasts, which represent in vitro models of ATRA induced granulocytic maturation of acute myelogenous leukemia (AML). In NB4 cells, PICLAMILAST accelerates the process of morphological granulocytic maturation and enhances the ATRA-dependent induction of specific differentiation markers such as NBT-reductase (NBTR) as well as CD11b. PICLAMILAST not only enhances, but also accelerates the process of granulocytic maturation set in motion by ATRA in acute myelogenous leukemia cells, reducing the time necessary to expose cells to ATRA to obtain maximal differentiation. Moreover, the compound increases the growth inhibitory effect of the retinoid in an additive fashion. PICLAMILAST treatment of NB4 cells results in a significant increase in the amounts of intracellular cAMP and cAMP-dependent protein kinase (PKA) over what observed in basal conditions. Neither basal nor PICLAMILAST-induced levels of cAMP and PKA are modulated by ATRA. PICLAMILAST exerts differential effects on a number of transcriptional factors involved in the process of leukemic cell maturation triggered by ATRA. In combination with the retinoid: I) it enhances the ligand-dependent transcriptional activity of the retinoic acid receptor, RARalpha, but not that of PML-RARalpha, the abnormal fusion product selectively expressed in acute promyelocytic leukemia blasts and NB4 cells. The phenomenon is associated with a PKA-dependent phosphorylation of RARalpha, which is activated by the PDE4 inhibitor, II) it causes an increase in the amounts of cEBPalpha as well as in the amounts and the activation state (tyrosine phosphorylation) of STAT1; III) it has no significant effect on the upregulation of cEBPepsilon. The direct modulation of RARalpha may underlie the enhancing action of PICLAMILAST on the expression of numerous ATRA-dependent genes, including cathepsin D and syaloadhesin. The PICLAMILAST dependent enhancement of the ATRA-dependent induction of NBT-R is suppressed by the cAMP antagonist, Rp-8Br-cAMP, and the specific PKA inhibitor H-89. However, H89 does not have the same effect on all the ATRA-dependent genes whose expression is super-induced by the PDE4 inhibitor. This indicates that PKA is not a necessary mediator of the interaction between PICLAMILAST and ATRA in myeloid cells. Surprisingly, treatment with PICLAMILAST results in a down-regulatory action on the phosphorylation state of the cAMP dependent CREBP transcriptional factor, which is highly active in undifferentiated NB4 cells. This phenomenon is more evident when cells are treated with combinations of ATRA and the PDE4 inhibitor. Interestingly, in NB4 cells, PICLAMILAST does not modulate the expression of cAMP- and CREBP-dependent genes, such as vinculin. Most importantly, the combination of PICLAMILAST+ATRA is more effective than the single components on the survival of SCID mice transplanted with NB4 cells. This altogether could represent a clinically relevant finding as it may represent a useful strategy to increase the therapeutic index of ATRA by decreasing its local and systemic toxicity.

1.-38. (canceled)

39. A method of treating neoplasms of lymphoid cells in a mammal, comprising administering to said mammal a therapeutically effective amount of a compound selected from the group consisting of
N-(3,5-dichloropyrid-4-yl)-3-cyclopentyloxy-4-methoxybenzamide [INN: PICLAMILAST], 3-cyclopropylmethoxy-4-difluoromethoxy-N-(3,5-dichloropyrid-4-yl)-benzamide [INN: ROFLUMILAST], 3-cyclopropylmethoxy-4-difluoromethoxy-N-(3,5-dichloro-1-oxy-pyrid-4-yl)-benzamide (Roflumilast-N-Oxide), 3-[3-(cyclopentyloxy)-4-methoxybenzyl]-6-(ethylamino)-8-isopropyl-3H-purine [Research Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research Code: CI-1018), 3,7-dihydro-3-(4-chlorophenyl)-1-propyl-1H-purine-2,6-dione [INN: AROFYLLINE], N-(3,5-dichloro-4-pyridinyl)-2-[1-(4-fluorobenzyl)-5-hydroxy-1H-indol-3-yl]-2-oxoacetamide [Research Code: AWD-12-281], N-(3,5-dichloropyridin-4-yl)-2-[5-fluoro-1-(4-fluorobenzyl)-1H-indol-3-yl]-2-oxoacetamide [Research Code: AWD-12-343], Tetrahydro-5-[4-methoxy-3-[(1S,2S,4R)-2-norbornyloxy]phenyl]-2(1H)-pyrimidone [INN: ATIZORAM]; β-[3-(cyclopentyloxy)-4-methoxyphenyl]-1,3-dihydro-1,3-dioxo-2H-isoindole-2-propanamide [Research Code: CDC-801], Methanesulfonic acid 2-(2,4-dichlorophenylcarbonyl)-3-ureidobenzo-furan-6-yl ester [INN: LIRIMILAST], 3,5-dichloro-4-[8-methoxy-2-(trifluoromethyl)quinolin-5-ylcarbox-amido]pyridine-1-oxide [Research Code: SCH-351591], cis-4-cyano-4-[3-cyclopentyloxy-4-methoxyphenyl]cyclohexane-1-carboxylic acid [INN: Cilomilast],
a compound with the research code CDC-998, D-4396, IC-485, CC-1088 or KW4490,
and pharmaceutically acceptable salts thereof.

40. A method for treating neoplasms of lymphoid cells in a mammal, comprising administering to said mammal therapeutically effective amounts of
(i) a compound selected from the group consisting of N-(3,5-dichloropyrid-4-yl)-3-cyclopentyloxy-4-methoxybenzamide [INN: PICLAMILAST], 3-cyclopropylmethoxy-4-difluoromethoxy-N-(3,5-dichloropyrid-4-yl)-benzamide [INN: ROFLUMILAST], 3-cyclopropylmethoxy-4-difluoromethoxy-N-(3,5-dichloro-1-oxy-pyrid-4-yl)-benzamide (Roflumilast-N-Oxide), 3-[3-(cyclopentyloxy)-4-methoxybenzyl]-6-(ethylamino)-8-isopropyl-3H-purine [Research Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research Code: CI-1018], 3,7-dihydro-3-(4-chlorophenyl)-1-propyl-1H-purine-2,6-dione [INN: AROFYLLINE], N-(3,5-dichloro-4-pyridinyl)-2-[1-(4-fluorobenzyl)-5-hydroxy-1H-indol-3-yl]-2-oxoacetamide [Research Code: AWD-12-281], N-(3,5-dichloropyridin-4-yl)-2-[5-fluoro-1-(4-fluorobenzyl)-1H-indol-3-yl]-2-oxoacetamide [Research Code: AWD-12-343], Tetrahydro-5-[4-methoxy-3-[(1S,2S,4R)-2-norbornyloxy]phenyl]-2(1H)-pyrimidone [INN: ATIZORAM]; β-[3-(cyclopentyloxy)-4-methoxyphenyl]-1,3-dihydro-1,3-dioxo-2H-isoindole-2-propanamide [Research Code: CDC-801], Methanesulfonic acid 2-(2,4-dichlorophenylcarbonyl)-3-ureidobenzo-furan-6-yl ester [INN: LIRIMILAST], 3,5-dichloro-4-[8-methoxy-2-(trifluoromethyl)quinolin-5-ylcarbox-amido]pyridine-1-oxide [Research Code: SCH-351591], cis-4-cyano-4-[3-cyclopentyloxy-4-methoxyphenyl]cyclohexane-1-carboxylic acid [INN: Cilomilast],
a compound with the research code CDC-998, D-4396, IC-485, CC-1088 or KW4490,
and pharmaceutically acceptable salts thereof,
and (ii) one or more differentiation inducing agents and/or an agent effective in raising intracellular concentrations of cAMP or a stable analogue thereof.

41. A method for treating neoplasms of lymphoid cells in a mammal, comprising administering to said mammal therapeutically effective amounts of
(i) a compound selected from the group consisting of N-(3,5-dichloropyrid-4-yl)-3-cyclopentyloxy-4-methoxybenzamide [INN: PICLAMILAST], 3-cyclopropylmethoxy-4-difluoromethoxy-N-(3,5-dichloropyrid-4-yl)-benzamide [INN: ROFLUMILAST], 3-cyclopropylmethoxy-4-difluoromethoxy-N-(3,5-dichloro-1-oxy-pyrid-4-yl)-benzamide (Roflumilast-N-Oxide), 3-[3-(cyclopentyloxy)-4-methoxybenzyl]-6-(ethylamino)-8-isopropyl-3H-purine [Research Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research Code: CI-1018], 3,7-dihydro-3-(4-chlorophenyl)-1-propyl-1H-purine-2,6-dione [INN: AROFYLLINE], N-(3,5-dichloro-4-pyridinyl)-2-[1-(4-fluorobenzyl)-5-hydroxy-1H-indol-3-yl]-2-oxoacetamide [Research Code: AWD-12-281], N-(3,5-dichloropyridin-4-yl)-2-[5-fluoro-1-(4-fluorobenzyl)-1H-indol-3-yl]-2-oxoacetamide [Research Code: AWD-12-343], Tetrahydro-5-[4-methoxy-3-[(1S,2S,4R)-2-norbornyloxy]phenyl]-2(1H)-pyrimidone [INN: ATIZORAM]; β-[3-(cyclopentyloxy)-4-methoxyphenyl]-1,3-dihydro-1,3-dioxo-2H-isoindole-2-propanamide [Research Code: CDC-801], Methanesulfonic acid 2-(2,4-dichlorophenylcarbonyl)-3-ureidobenzo-furan-6-yl ester [INN: LIRIMILAST], 3,5-dichloro-4-[8-methoxy-2-(trifluoromethyl)quinolin-5-ylcarbox-amido]pyridine-1-oxide [Research Code: SCH-351591], cis-4-cyano-4-[3-cyclopentyloxy-4-methoxyphenyl]cyclohexane-1-carboxylic acid [INN: Cilomilast],
a compound with the research code CDC-998, D-4396, IC-485, CC-1088 or KW4490,
and pharmaceutically acceptable salts thereof,
and (ii) one or more differentiation inducing agents.

42. A method for treating neoplasms of lymphoid cells in a mammal, comprising administering to said mammal therapeutically effective amounts of
(i) a compound selected from the group consisting of N-(3,5-dichloropyrid-4-yl)-3-cyclopentyloxy-4-methoxybenzamide [INN: PICLAMILAST], 3-cyclopropylmethoxy-4-difluoromethoxy-N-(3,5-dichloropyrid-4-yl)-benzamide [INN: ROFLUMILAST], 3-cyclopropylmethoxy-4-difluoromethoxy-N-(3,5-dichloro-1-oxy-pyrid-4-yl)-benzamide (Roflumilast-N-Oxide), 3-[3-(cyclopentyloxy)-4-methoxybenzyl]-6-(ethylamino)-8-isopropyl-3H-purine [Research Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research Code: CI-1018], 3,7-dihydro-3-(4-chlorophenyl)-1-propyl-1H-purine-2,6-dione [INN: AROFYLLINE], N-(3,5-dichloro-4-pyridinyl)-2-[1-(4-fluorobenzyl)-5-hydroxy-1H-indol-3-yl]-2-oxoacetamide [Research Code: AWD-12-281], N-(3,5-dichloropyridin-4-yl)-2-[5-fluoro-1-(4-fluorobenzyl)-1H-indol-3-yl]-2-oxoacetamide [Research Code: AWD-12-343], Tetrahydro-5-[4-methoxy-3-[(1S,2S,4R)-2-norbornyloxy]phenyl]-2(1H)-pyrimidone [INN: ATIZORAM]; β-[3-(cyclopentyloxy)-4-methoxyphenyl]-1,3-dihydro-1,3-dioxo-2H-isoindole-2-propanamide [Research Code: CDC-801], Methanesulfonic acid 2-(2,4-dichlorophenylcarbonyl)-3-ureidobenzo-furan-6-yl ester [INN: LIRIMILAST], 3,5-dichloro-4-[8-methoxy-2-(trifluoromethyl)quinolin-5-ylcarbox-amido]pyridine-1-oxide [Research Code: SCH-351591], cis-4-cyano-4-[3-cyclopentyloxy-4-methoxyphenyl]cyclohexane-1-carboxylic acid [INN: Cilomilast],
a compound with the research code CDC-998, D-4396, IC-485, CC-1088 or KW4490,
and pharmaceutically acceptable salts thereof,
and (ii) an agent effective in raising intracellular concentrations of cAMP or a stable analogue thereof.

43. (canceled)

44. The method according to claim 39, wherein the compound of component (i) is selected from the group consisting of N-(3,5-dichloro-4-pyridinyl)-2-[1-(4-fluorobenzyl)-5-hydroxy-1H-indol-3-yl]-2-oxoacetamide [Research Code: AWD-12-281], cis-4-cyano-4-[3-cyclopentyloxy-4-methoxyphenyl]cyclohexane-1-carboxylic acid [INN: Cilomilast], 3-cyclopropylmethoxy-4-difluoromethoxy-N-(3,5-dichloropyrid-4-yl)-benzamide [INN: ROFLUMILAST], 3-cyclopropylmethoxy-4-difluoromethoxy-N-(3,5-dichloro-1-oxy-pyrid-4-yl)-benzamide (Roflumilast-N-Oxide) and pharmaceutically acceptable salts thereof.

45. The method according to claim 39, wherein the compound of component (i) is selected from the group consisting of 3-cyclopropylmethoxy-4-difluoromethoxy-N-(3,5-dichloropyrid-4-yl)-benzamide [INN: ROFLUMILAST], 3-cyclopropylmethoxy-4-difluoromethoxy-N-(3,5-dichloro-1-oxy-pyrid-4-yl)-benzamide (Roflumilast-N-Oxide) and pharmaceutically acceptable salts thereof.

46. The method according to claim 39, wherein the compound of component (i) is 3-cyclopropylmethoxy-4-difluoromethoxy-N-(3,5-dichloropyrid-4-yl)-benzamide [INN: ROFLUMILAST] or a pharmaceutically acceptable salt thereof.

47. The method according to claim 39, wherein the compound of component (i) is 3-cyclopropylmethoxy-4-difluoromethoxy-N-(3,5-dichloro-1-oxy-pyrid-4-yl)-benzamide (Roflumilast-N-Oxide) or a pharmaceutically acceptable salt thereof.

48. The method according to claim 39, wherein the compound of component (i) is N-(3,5-dichloro-4-pyridinyl)-2-[1-(4-fluorobenzyl)-5-hydroxy-1H-indol-3-yl]-2-oxoacetamide [Research Code: AWD-12-281] or a pharmaceutically acceptable salt thereof.

49. The method according to claim 39, wherein the compound of component (i) is cis-4-cyano-4-[3-cyclopentyloxy-4-methoxyphenyl]cyclohexane-1-carboxylic acid [INN: Cilomilast] or a pharmaceutically acceptable salt thereof.

50.-61. (canceled)

62. A treatment combination for neoplasms of lymphoid cells, comprising: therapeutically effective amounts of
(i) a compound selected from the group consisting of N-(3,5-dichloropyrid-4-yl)-3-cyclopentyloxy-4-methoxybenzamide [INN: PICLAMILAST], 3-cyclopropylmethoxy-4-difluoromethoxy-N-(3,5-dichloropyrid-4-yl)-benzamide [INN: ROFLUMILAST], 3-cyclopropylmethoxy-4-difluoromethoxy-N-(3,5-dichloro-1-oxy-pyrid-4-yl)-benzamide (Roflumilast-N-Oxide), 3-[3-(cyclopentyloxy)-4-methoxybenzyl]-6-(ethylamino)-8-isopropyl-3H-purine [Research Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research Code: CI-1018], 3,7-dihydro-3-(4-chlorophenyl)-1-propyl-1H-purine-2,6-dione [INN: AROFYLLINE], N-(3,5-dichloro-4-pyridinyl)-2-[1-(4-fluorobenzyl)-5-hydroxy-1H-indol-3-yl]-2-oxoacetamide [Research Code: AWD-12-281], N-(3,5-dichloropyridin-4-yl)-2-[5-fluoro-1-(4-fluorobenzyl)-1H-indol-3-yl]-2-oxoacetamide [Research Code: AWD-12-343], Tetrahydro-5-[4-methoxy-3-[(1S,2S,4R)-2-norbornyloxy]phenyl]-2(1H)-pyrimidone [INN: ATIZORAM]; β-[3-(cyclopentyloxy)-4-methoxyphenyl]-1,3-dihydro-1,3-dioxo-2H-isoindole-2-propanamide [Research Code: CDC-801], Methanesulfonic acid 2-(2,4-dichlorophenylcarbonyl)-3-ureidobenzo-furan-6-yl ester [INN: LIRIMILAST], 3,5-dichloro-4-[8-methoxy-2-(trifluoromethyl)quinolin-5-ylcarbox-amido]pyridine-1-oxide [Research Code: SCH-351591], cis-4-cyano-4-[3-cyclopentyloxy-4-methoxyphenyl]cyclohexane-1-carboxylic acid [INN: Cilomilast],
a compound with the research code CDC-998, D-4396, IC-485, CC-1088 or KW4490
and pharmaceutically acceptable salts thereof,
and (ii) one or more differentiation inducing agents and/or an agent effective in raising intracellular concentrations of cAMP or a stable analogue of cAMP.

63. A treatment combination for neoplasms of lymphoid cells, comprising: therapeutically effective amounts of
(i) a compound selected from the group consisting of N-(3,5-dichloropyrid-4-yl)-3-cyclopentyloxy-4-methoxybenzamide [INN: PICLAMILAST], 3-cyclopropylmethoxy-4-difluoromethoxy-N-(3,5-dichloropyrid-4-yl)-benzamide [INN: ROFLUMILAST], 3-cyclopropylmethoxy-4-difluoromethoxy-N-(3,5-dichloro-1-oxy-pyrid-4-yl)-benzamide (Roflumilast-N-Oxide), 3-[3-(cyclopentyloxy)-4-methoxybenzyl]-6-(ethylamino)-8-isopropyl-3H-purine [Research Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research Code: CI-1018], 3,7-dihydro-3-(4-chlorophenyl)-1-propyl-1H-purine-2,6-dione [INN: AROFYLLINE], N-(3,5-dichloro-4-pyridinyl)-2-[1-(4-fluorobenzyl)-5-hydroxy-1H-indol-3-yl]-2-oxoacetamide [Research Code: AWD-12-281], N-(3,5-dichloropyridin-4-yl)-2-[5-fluoro-1-(4-fluorobenzyl)-1H-indol-3-yl]-2-oxoacetamide [Research Code: AWD-12-343], Tetrahydro-5-[4-methoxy-3-[(1S,2S,4R)-2-norbornyloxy]phenyl]-2(1H)-pyrimidone [INN: ATIZORAM]; β-[3-(cyclopentyloxy)-4-methoxyphenyl]-1,3-dihydro-1,3-dioxo-2H-isoindole-2-propanamide [Research Code: CDC-801], Methanesulfonic acid 2-(2,4-dichlorophenylcarbonyl)-3-ureidobenzo-furan-6-yl ester [INN: LIRIMILAST], 3,5-dichloro-4-[8-methoxy-2-(trifluoromethyl)quinolin-5-ylcarbox-amido]pyridine-1-oxide [Research Code: SCH-351591], cis-4-cyano-4-[3-cyclopentyloxy-4-methoxyphenyl]cyclohexane-1-carboxylic acid [INN: Cilomilast],
a compound with the research code CDC-998, D-4396, IC-485, CC-1088 or KW4490
and pharmaceutically acceptable salts thereof,
and (ii) one or more differentiation inducing agents.

64. A treatment combination for neoplasms of lymphoid cells, comprising: therapeutically effective amounts of
(i) a compound selected from the group consisting of N-(3,5-dichloropyrid-4-yl)-3-cyclopentyloxy-4-methoxybenzamide [INN: PICLAMILAST], 3-cyclopropylmethoxy-4-difluoromethoxy-N-(3,5-dichloropyrid-4-yl)-benzamide [INN: ROFLUMILAST], 3-cyclopropylmethoxy-4-difluoromethoxy-N-(3,5-dichloro-1-oxy-pyrid-4-yl)-benzamide (Roflumilast-N-Oxide), 3-[3-(cyclopentyloxy)-4-methoxybenzyl]-6-(ethylamino)-8-isopropyl-3H-purine [Research Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research Code: CI-1018], 3,7-dihydro-3-(4-chlorophenyl)-1-propyl-1H-purine-2,6-dione [INN: AROFYLLINE], N-(3,5-dichloro-4-pyridinyl)-2-[1-(4-fluorobenzyl)-5-hydroxy-1H-indol-3-yl]-2-oxoacetamide [Research Code: AWD-12-281], N-(3,5-dichloropyridin-4-yl)-2-[5-fluoro-1-(4-fluorobenzyl)-1H-indol-3-yl]-2-oxoacetamide [Research Code: AWD-12-343], Tetrahydro-5-[4-methoxy-3-[(1S,2S,4R)-2-norbornyloxy]phenyl]-2(1H)-pyrimidone [INN: ATIZORAM]; β-[3-(cyclopentyloxy)-4-methoxyphenyl]-1,3-dihydro-1,3-dioxo-2H-isoindole-2-propanamide [Research Code: CDC-801], Methanesulfonic acid 2-(2,4-dichlorophenylcarbonyl)-3-ureidobenzo-furan-6-yl ester [INN: LIRIMILAST], 3,5-dichloro-4-[8-methoxy-2-(trifluoromethyl)quinolin-5-ylcarbox-amido]pyridine-1-oxide [Research Code: SCH-351591], cis-4-cyano-4-[3-cyclopentyloxy-4-methoxyphenyl]cyclohexane-1-carboxylic acid [INN: Cilomilast],
a compound with the research code CDC-998, D-4396, IC-485, CC-1088 or KW4490
and pharmaceutically acceptable salts thereof,
and (ii) an agent effective in raising intracellular concentrations of cAMP or a stable analogue of cAMP.

65. The treatment combination according to claim 62, wherein the compound of component (i) is selected from the group consisting of
N-(3,5-dichloro-4-pyridinyl)-2-[1-(4-fluorobenzyl)-5-hydroxy-1H-indol-3-yl]-2-oxoacetamide [Research Code: AWD-12-281], cis-4-cyano-4-[3-cyclopentyloxy-4-methoxyphenyl]cyclohexane-1-carboxylic acid [INN: Cilomilast], 3-cyclopropylmethoxy-4-difluoromethoxy-N-(3,5-dichloropyrid-4-yl)-benzamide [INN: ROFLUMILAST], 3-cyclopropylmethoxy-4-difluoromethoxy-N-(3,5-dichloro-1-oxy-pyrid-4-yl)-benzamide (Roflumilast-N-Oxide) and pharmaceutically acceptable salts thereof.

66. The treatment combination according to claim 62, wherein the compound of component (i) is selected from the group consisting of 3-cyclopropylmethoxy-4-difluoromethoxy-N-(3,5-dichloropyrid-4-yl)-benzamide (INN: ROFLUMILAST], 3-cyclopropylmethoxy-4-difluoromethoxy-N-(3,5-dichloro-1-oxy-pyrid-4-yl)-benzamide (Roflumilast-N-Oxide) and pharmaceutically acceptable salts thereof.

67. The treatment combination according to claim 62, wherein the compound of component (i) is 3-cyclopropylmethoxy-4-difluoromethoxy-N-(3,5-dichloropyrid-4-yl)-benzamide [INN: ROFLUMILAST] or a pharmaceutically acceptable salt thereof.

68. The treatment combination according to claim 62, wherein the compound of component (i) is 3-cyclopropylmethoxy-4-difluoromethoxy-N-(3,5-dichloro-1-oxy-pyrid-4-yl)-benzamide (Roflumilast-N-Oxide) or a pharmaceutically acceptable salt thereof.

69. The treatment combination according to claim 62, wherein the compound of component (i) is N-(3,5-dichloro-4-pyridinyl)-2-[1-(4-fluorobenzyl)-5-hydroxy-1H-indol-3-yl]-2-oxoacetamide [Research Code: AWD-12-281] or a pharmaceutically acceptable salt thereof.

70. The treatment combination according to claim 62, wherein the compound of component (i) is cis-4-cyano-4-[3-cyclopentyloxy-4-methoxyphenyl]cyclohexane-1-carboxylic acid [INN: Cilomilast] or a pharmaceutically acceptable salt thereof.

71.-74. (canceled)

75. The method according to claim 40, wherein the differentiation inducing agent is selected from the group consisting of all trans retinoic acid, 13-cis-retinoic acid, CD437, rexinoids, histone deacetylase inhibitors, DNA methyltransferase inhibitors, hematopoietic growth factors, interferon α, interleukin 1, TRAIL, hexamethylene bisacetamide, cholecalciferol, arsenic trioxide, green tea catechin epigallocatechin-3-gallate, DNA topoisomerase II inhibitors, taraxinic acid, verticinone, PPAR-gamma agonists, antibodies versus CD19, CD20 or CD22, CD33-antibodies alone or as conjugate, alkylating cytostatika, purine analogs, cytosine-arabinosides, anticylines, vinca-alkaloids and glucocorticosteroids.

76. The method according to claim 40, wherein the differentiation inducing agent is a histone deacetylase inhibitor.

77. The method according to claim 40, wherein the differentiation inducing agent is all trans retinoic acid.

78. The method according to claim 40, wherein the agent effective in raising intracellular concentrations of cAMP is selected from the group consisting of prostaglandin E2, prostacyclin derivatives, dopamine, dobutamine, β2-adrenoreceptor agonists, adenosine A1 receptor agonists, adenosine A2 receptor agonists and forskolin.

79. A treatment combination according to claim 62, wherein the differentiation inducing agent is selected from the group consisting of all trans retinoic acid, 13-cis-retinoic acid, CD437, rexinoids, histone deacetylase inhibitors, DNA methyltransferase inhibitors, hematopoietic growth factors, interferon α, interleukin 1, TRAIL, hexamethylene bisacetamide, cholecalciferol, arsenic trioxide, green tea catechin epigallocatechin-3-gallate, DNA topoisomerase II inhibitors, taraxinic acid, verticinone, PPAR-gamma agonists, antibodies versus CD19, CD20 or CD22, CD33-antibodies alone or as conjugate, alkylating cytostatika, purine analogs, cytosine-arabinosides, anticylines, vinca-alkaloids and glucocorticosteroids.

80. A treatment combination according to claim 62, wherein the agent effective in raising intracellular concentrations of cAMP is selected from the group consisting of prostaglandin E2, prostacyclin derivatives, dopamine, dobutamine, β2-adrenoreceptor agonists, adenosine A1 receptor agonists, adenosine A2 receptor agonists and forskolin.

81. A treatment combination according to claim 62, wherein the differentiation inducing agent is a histone deacetylase inhibitor.

82. A treatment combination according to claim 62, wherein the differentiation inducing agent is all trans retinoic acid.

83. (canceled)

84. The method according to claim 39, wherein the neoplasm of lymphoid cells is leukemia.

85. The treatment combination according to claim 62, wherein the neoplasm of lymphoid cells is leukemia.
